(19) United States
(12) Patent Application Publication
(10) Pub. No.: US 2008/0073201 A1
(43) Pub. Date: Mar. 27, 2008

(54) Acetonitrile Recycling Process

(75) Inventors: August Van Gysel, Solre le Chateau (FR); Frans Soeterbroeck, Zwijndrecht (BE); Geert Vermeulen, Ramsel (BE)

Correspondence Address: NIXON & VANDERHYE, PC, 901 NORTH GLEBE ROAD, 11TH FLOOR, ARLINGTON, VA, 22203, US

(73) Assignee: UCB, S.A., Brussels (BE)

(21) Appl. No.: 10/578,264

(22) PCT Filed: Oct. 28, 2004
(86) PCT No.: PCT/EP04/12167

§ 371 Date: Aug. 2, 2007

(30) Foreign Application Priority Data
Nov. 4, 2003 (EP) 03025184.7

(51) Int. Cl. C07C 253/34; B01D 3/36; C07C 255/03; B01D 61/36

(52) U.S. Cl. 203/34; 203/39; 203/41

(57) ABSTRACT

The present invention relates to improved processes to recover acetonitrile from a diluted aqueous low grade acetonitrile feedstock.

The present invention concerns an improved process to recover acetonitrile from a diluted aqueous low grade acetonitrile feedstock.

The process comprises a first azeotropic distillation and a second azeotropic distillation, wherein the water content in the stream is reduced to below 16% by weight before it is fed to the distillation column in which the second distillation is performed.

In order to achieve this, in one embodiment, the first azeotropic distillation is followed by pervaporation.

In a second embodiment the water content is reduced by use of a pressure-swing distillation.

There are different types of acetonitrile currently used in the marketplace:

Low grade acetonitrile comprises HPLC purification process waste such as from the purification of peptides, DNA synthesis process waste, and pharmaceutical drug manufacturing process waste. Low grade acetonitrile contains between about 35% by weight to 85% by weight acetonitrile and less than approximately 40% of water.

Industrial grade acetonitrile is at least 99.75% by weight acetonitrile, contains approximately 500 ppm of water and is typically used in gas chromatography applications and agricultural pesticide manufacturing processes.

HPLC grade acetonitrile is a high purity acetonitrile containing at least 99.8% by weight acetonitrile, a U.V. transmission spectrum measurement of more than 90% at a 220 nm wavelength and more than 98% at a 240 nm wavelength, and can contain more than 100 ppm of water.

Furthermore there still is DNA synthesis grade acetonitrile with a purity of at least 99.9% by weight acetonitrile, containing approximately 50 ppm or less of water, and ultra-pure acetonitrile with a purity of at least 99.99% containing approximately 20 ppm or less of water.

There exist prior methods of purifying acetonitrile from industrial or higher grade acetonitrile feedstocks. These methods include treatment by various absorbents to remove organic impurities, e.g. by use of active alumina, active bauxite, active carbon, special aluminosilicates or molecular sieves, and treatments to remove water, e.g. by use of active alumina, calcium chloride desiccants, silica gel, aluminosilicates or molecular sieves. Other methods of purifying acetonitrile from industrial or higher grades of acetonitrile have included the use of oxidising agents such as air, oxygen or ozone, followed by distillation and/or absorption to remove the oxidized impurities, see U.S. Pat. No. 5,426,208.

None of the above described methods is applicable to recover acetonitrile from a diluted aqueous low grade acetonitrile feedstock. Low grade acetonitrile waste streams are therefore usually being disposed of by incineration.

The literature reveals one document, U.S. Pat. No. 6,395,142 that describes a purification process for low grade acetonitrile feedstocks. 3 different sources of low-grade acetonitrile feedstocks are mentioned in this patent: DNA synthesis process waste, HPLC process waste and pharmaceutical drug manufacturing waste. Low-grade acetonitrile is described in this patent as a feedstock containing primarily 30 to 85% by weight of acetonitrile and containing less than approximately 40% by weight of water.

The process consists of two consecutive distillations. The low grade feedstock is fed to a first distillation column and the acetonitrile and a first set of impurities with a lower boiling point than acetonitrile is separated from a 2^{nd} set of impurities having a higher boiling point than acetonitrile, the acetonitrile and the first set of impurities being drawn as a vapor from said first distillation column, the 2^{nd} set of impurities being produced as the first distillation column bottoms.

The acetonitrile and the first set of impurities are then condensed and fed to a 2^{nd} distillation column, separating the first set of impurities from the acetonitrile being produced as the second distillation column bottom.

Although U.S. Pat. No. 6,395,142 states that the process is applicable on all 3 low grade acetonitrile sources, thus feedstocks containing primarily 30 to 85% by weight of acetonitrile and containing less than approximately 40% by weight of water, it is evident that the process described in the patent is only efficient if the feedstock
either contains less than 16% by weight of water, as the acetonitrile/water azeotrope is approximately 16% by weight water and 84% by weight acetonitrile, and preferably less than 5% by weight and more preferably less than 2% by weight water. This is indicated in U.S. Pat. No. 6,395,142 in column 4 lines 19-24; in the example in column 7 line 15; in column 8 lines 62-64; in claims 15, 16 and 17.
or contains other constituents forming azeotropes with water with a lower boiling point than the acetonitrile/water azeotrope (76° C.). Such constituents are for example tetrahydrofuran (THF) (THF/water azeotrope % weight/weight 96/4 boils at 64° C.) or dichloromethane (DCM) (DCM/water azeotrope % weight/weight 99/1 boils at 38° C.). The presence of such constituents is indicated in U.S. Pat. No. 6,395,142 (column 4 lines 46-47; column 5 lines 16-17; table 1; table 4; claims 18, 27, 46, and 56)

If, beside acetonitrile, no other constituents forming azeotropes with water and having a lower boiling point than the acetonitrile/water azeotrope would be present, the initial water concentration in the feedstock may not be too high. Otherwise it would be impossible in the 2^{nd} distillation to obtain in an efficient and economic way a high grade acetonitrile as column bottom. The higher the initial concentration of water the more acetonitrile would have to be evaporated as a acetonitrile/water azeotrope in the 2^{nd} distillation column to obtain a 2^{nd} distillation column bottom containing less than 0.1 weight % water and to meet the HPLC grade acetonitrile specification for water. Thus the less other such constituents are present, the lower the water concentration in the initial feedstock has to be. If no such constituents are present, the water concentration in the initial feedstock has to be as low as possible in order to have an economical process.

Based on these considerations, it is clear that the prior art process is not efficient for all low grade feedstocks defined in the patent as feedstocks containing primarily 30 to 85% by weight of acetonitrile and containing approximately 40% by weight of water. For such high water concentrations the process can never be economically viable.

In summary, the process described in U.S. Pat. No. 6,395,142 B1 only permits the treatment of a low grade acetonitrile feedstock containing as little water as possible, or containing other constituents forming azeotropes with water with a lower boiling point than the acetonitrile/water azeotrope (76° C.). If no such constituents are present, for each percent of water, 84/16% or 5.25% of acetonitrile would be lost in the 2^{nd} distillation to get a pure acetonitrile bottom containing less than 0.1% of water. It is evident that for a source containing 16% of water, all the acetonitrile would have to be distilled in the 2^{nd} column, leaving no pure acetonitrile anymore in the column bottom.

Hence, there is a need to develop a process which is capable of efficiently recovering acetonitrile from a diluted aqueous low grade acetonitrile feedstock, wherein the feedstock may contain 16% by weight or even 50% by weight water and more.

This technical problem is solved by the processes of the present invention. Processes are provided that allow to reduce the water content of the stream that is fed to the distillation column in which the second distillation is performed, to below 16% by weight. Thereby it is avoided that during the second distillation the entire amount of acetonitrile is distilled in the form of the acetonitrile/water azeotrope. In one embodiment this has been achieved by the combination of distillation and pervaporation. In a second embodiment the reduction in water content is achieved by using pressure-swing distillation wherein the first distillation is performed at below atmospheric pressure and the second distillation is performed at atmospheric pressure.

Both embodiments allow for efficient recovery of acetonitrile from low-grade acetonitrile feedstocks having a water content of 16% by weight or even 50% by weight or more. The feedstock to be used according to this invention may be any aqueous mixture containing acetonitrile, e.g. low grade acetonitrile waste from a HPLC purification process of peptides.

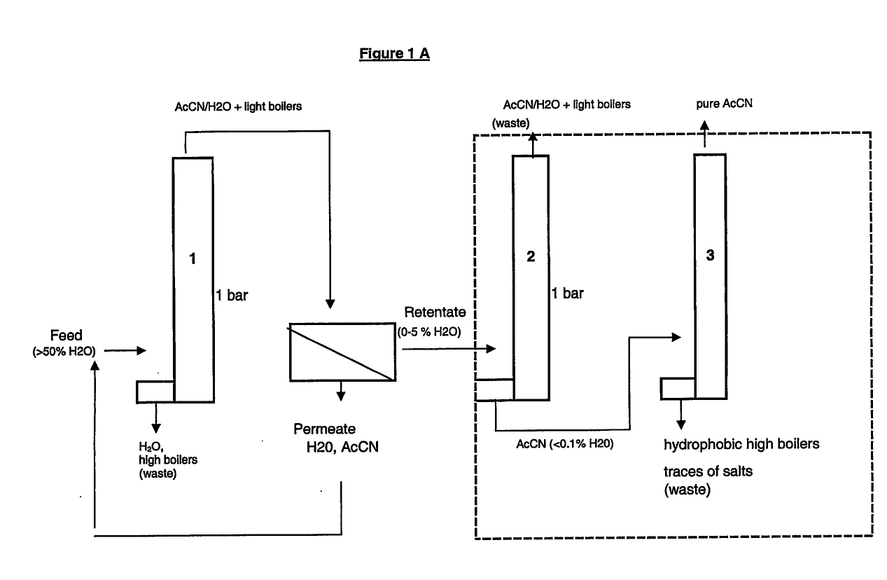

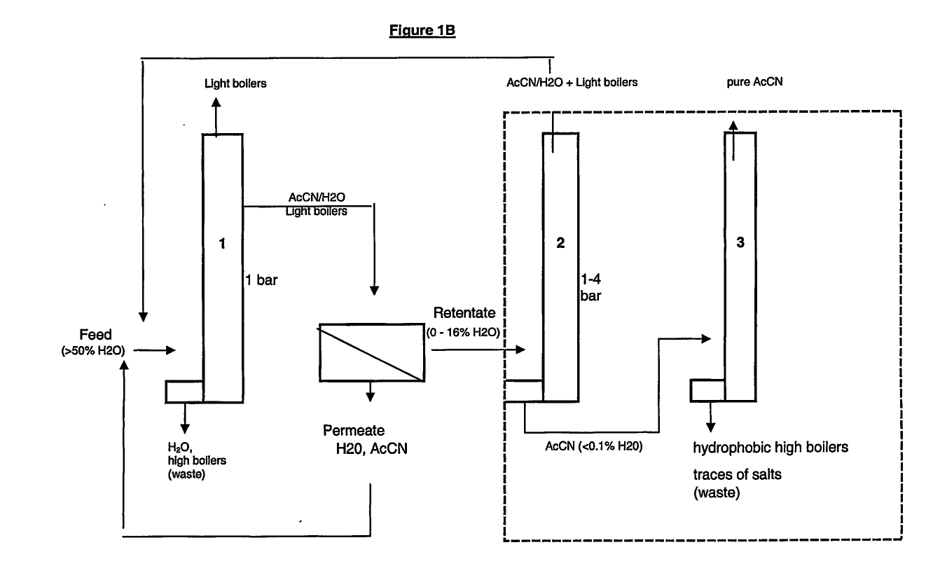

FIG. 1A represents schematically the process according to a preferred aspect of the first embodiment of the invention. The reference numbers 1, 2 and 3 represent three different distillations which are performed in sequence. Each distillation may be performed in a different column, as implied by FIG. 1A. Alternatively two or three distillations may be executed one after the other on the same column. In this case, reference numbers 1, 2 and 3 are to be understood as representing consecutive distillations, although not being performed in separate columns.

FIG. 1B represents schematically the process according to a preferred modification of the first embodiment of the invention. The reference numbers 1, 2 and 3 represent three different distillations which are performed in sequence. Each distillation may be performed in a different column, as implied by FIG. 1B. Alternatively two or three distillations may be executed one after the other on the same column. In this case, reference numbers 1, 2 and 3 are to be understood as representing consecutive distillations, although not being performed in separate columns.

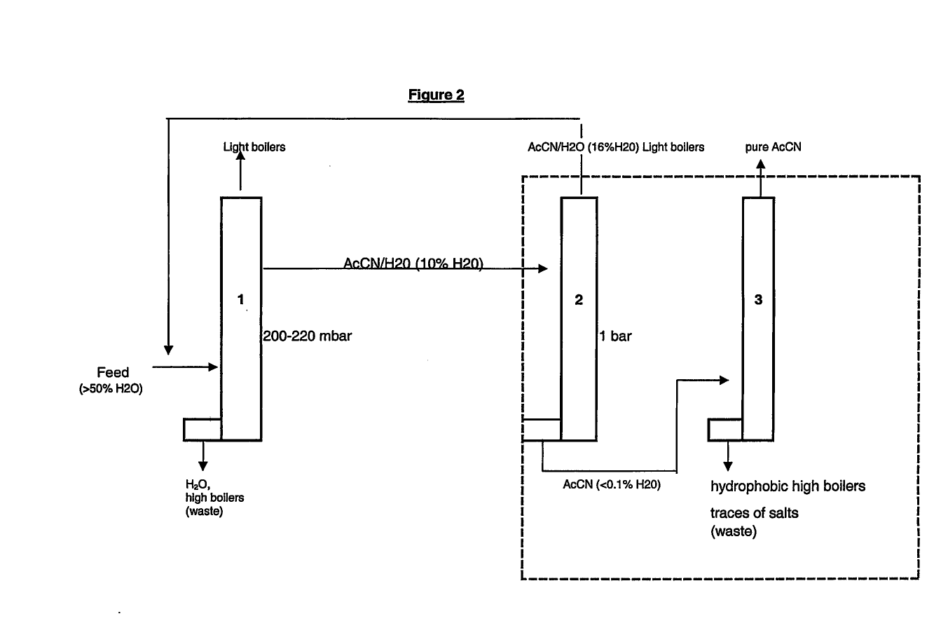

FIG. 2 represents schematically the process according to a preferred aspect of the second embodiment of the invention. The reference numbers 1, 2 and 3 represent three different distillations which are performed in sequence. Each distillation may be performed in a different column, as implied by FIG. 2. Alternatively two or three distillations may be executed one after the other on the same column. In this case, reference numbers 1, 2 and 3 are to be understood as representing consecutive distillations, although not being performed in separate columns.

In one embodiment, the invention therefore provides a process for purifying an acetonitrile feedstock comprising acetonitrile, 16 up to 90% by weight of water, low boiling impurities having a boiling temperature lower than the acetonitrile/water azeotrope boiling point, and high boiling impurities having a boiling temperature higher than the acetonitrile boiling point, the process comprising in sequence the steps of:
A) introducing the feedstock into a distillation column and, by performing a distillation, separating the acetonitrile/water azeotrope and the low boiling impurities from the high boiling impurities, the acetonitrile/water azeotrope and the low boiling impurities being drawn as a vapor from the top of said distillation column, the high boiling impurities being produced as the distillation column bottoms;
B) feeding the acetonitrile/water azeotrope and the low boiling impurities to a pervaporation unit capable of separating the water from the acetonitrile, the majority of water being collected as permeate from the pervaporation unit, and the acetonitrile, low boiling impurities and remaining water being collected as retentate;
C) introducing the retentate of the pervaporation unit to a distillation column and, by performing another distillation, withdrawing the remaining acetonitrile/water azeotrope and the low boiling impurities as a vapor from said distillation column, wherein the acetonitrile is recovered from the distillation column bottoms.

The acetonitrile/water azeotrope and the low boiling impurities that are drawn as vapor from the column after the distillation of step C, are considered as waste.

Those impurities having a boiling point between the acetonitrile/water azeotrope boiling point and the acetonitrile boiling point, will be withdrawn in step A as vapor together with the low boiling impurities and the acetonitrile/water azeotrope. In step C, depending on the distillation temperature selected and the exact boiling temperature of these impurities, they will mainly be withdrawn as vapor at the top of the column.

The distillations of steps A and C may be executed one after the other on the same column. However, it is preferable to perform them in separate columns. Hence, it is preferable to perform the distillation of step A in a first distillation column and the distillation of step C in a second distillation column.

According to this first embodiment, pervaporation is performed in order to reduce the water content of the stream fed to the column in which the distillation of step C is performed. Therefore, the acetonitrile/water azeotrope and the low boiling impurities that are drawn from the distillation column in which the distillation of step A takes place, are fed to a pervaporation unit, either in the form of vapor under pressure or, after a step of condensing, in the form of a condensate.

Pervaporation is an energy efficient combination of membrane permeation and evaporation. It is useful for the dehydration of organic solvents. Pervaporation involves the separation of two or more components across a membrane by differing rates of diffusion through a thin polymer and an evaporative phase change. A concentrate and vapor pressure gradient is used to allow one component to preferentially permeate across the membrane. A vacuum applied to the permeate side is coupled with the immediate condensation of the permeated vapors. It is preferred to have high selectivity through the membrane. In order to remove water from organic solutions, as done in the present invention, asymmetric, hydrophilic, dense and sufficiently cross-linked polymer membranes are used. Asymmetric means that the membrane is a multilayer membrane consisting of a support layer, an intermediate layer and a separating top-layer. These hydrophilic membranes are preferably selective for water. Dense means that the membrane has no pores, and sufficiently cross-linked means that the polymer membrane avoids excessive swelling in solvents. Examples are the membranes PERVAP® 2216, PERVAP® 2256 and PERVAP® 2201, all purchased from Sulzer. The most preferred membrane is the Sulzer PERVAP® 2201 membrane.

The term pervaporation is used when separating liquids. If a saturated vapour mixture is to be separated, the process is called vapourpermeation, although it is essentially the same.

When pervaporation is performed, the stream reaching the distillation column in which the distillation of step C is performed, has a water content lower than 5% by weight, more preferably lower than 2% by weight and most preferably lower than 0.1% by weight.

The term pervaporation unit is defined as the entire equipment needed to perform pervaporation and includes the feed pump, the feed preheater, different pervaporation modules, an interstage heat exchanger, the condenser, the vacuum pump and the permeate pump.

In a modified version of this first embodiment, the acetonitrile/water azeotrope leaves the column in which the distillation of step A is performed as a side draw, the majority of the light boiling impurities are withdrawn at the top of this column and considered as waste. In this modified version of the first embodiment, a smaller pervaporation unit is used since the feed for the column in which the distillation of step C is performed does not need to be reduced to 5% water content or less, instead the water content may be up to the level of the azeotrope composition (16% by weight). This is possible, because the acetonitrile/water azeotrope distillate of the distillation of step C is not discarded as in the first version of this embodiment described above, but it is recycled to the feedstock. The use of smaller pervaporation units is, due to the high prize of pervaporation membranes, very advantageous. In this modified version of the first embodiment, the surface of the pervaporation unit may be reduced to 30% of the surface used in the unmodified version of the first embodiment.

Hence, in a modified version of a first embodiment, this invention provides a process for purifying an acetonitrile feedstock comprising acetonitrile, 16% up to 90% by weight of water, low boiling impurities having a boiling temperature lower than the acetonitrile/water azeotrope boiling point, and high boiling impurities having a boiling temperature higher than the acetonitrile boiling point, the process comprising in sequence the steps of:
A′) introducing the feedstock into a distillation column and, by performing a distillation, separating the acetonitrile/water azeotrope and the low boiling impurities from the high boiling impurities, the high boiling impurities being produced as the distillation column bottoms, a majority of the low boiling impurities being drawn as a vapor from the top of said distillation column, and the acetonitrile/water azeotrope and a remaining part of the low boiling impurities being drawn as a side draw of that distillation column;
B′) feeding the acetonitrile/water azeotrope and the low boiling impurities to a pervaporation unit capable of separating the water from the acetonitrile, the majority of water being collected as permeate from the pervaporation unit, and the acetonitrile, low boiling impurities and remaining water being collected as retentate;
C′) introducing the retentate of the pervaporation unit into a distillation column and, by performing another distillation, withdrawing the acetonitrile/water azeotrope and the low boiling impurities as a vapor, wherein the acetonitrile is recovered from the distillation column bottoms.

It is preferable that the acetonitrile/water azeotrope leaving the column in which the distillation of step C′ is performed, is recycled to the acetonitrile feedstock and introduced into the distillation column, in which the distillation of step A′ is performed.

Distillation of step C′ can be performed at atmospheric pressure or at super atmospheric pressures if one wants to enlarge the water content of the acetonitrile/water azeotrope. A greater water content means a more economical distillation C′.

Preferably, the acetonitrile/water azeotrope leaving the column after the distillation of step A′ as a side draw, is in the form of a liquid.

Those impurities having a boiling point between the acetonitrile/water azeotrope boiling point and the acetonitrile boiling point, will be withdrawn in step A′ partly as vapor together with the low boiling impurities and partly as a liquid side draw together with the acetonitrile/water azeotrope. In step C′, depending on the distillation temperature selected and the exact boiling temperature of these impurities, they will mainly be withdrawn as vapor at the top of the column.

The distillations of steps A′ and C′ may be executed one after the other on the same column. However, it is preferable to perform them in separate columns. Hence, it is preferable to perform the distillation of step A′ in a first distillation column and the distillation of step C′ in a second distillation column.

In a second embodiment, this invention provides a process for purifying an acetonitrile feedstock comprising acetonitrile, 16% up to 90% by weight water, low boiling impurities having a boiling temperature lower than the acetonitrile/water azeotrope boiling point, and high boiling impurities having a boiling temperature higher than the acetonitrile boiling point, the process comprising in sequence the steps of:
A″) introducing the feedstock into a distillation column and, by performing a distillation at below atmospheric pressure, separating the acetonitrile/water azeotrope and the low boiling impurities from the high boiling impurities, the high boiling impurities being produced as distillation column bottoms, the total or majority of the low boiling impurities being drawn as vapor via the top of the distillation column and the acetonitrile/water azeotrope and potentially remaining low boiling impurities being drawn as a side draw of that distillation column
C″) introducing the acetonitrile/water azeotrope side draw into a distillation column and, by performing another distillation, at atmospheric pressure, enriching the acetonitrile/water azeotrope with water and withdrawing it as a vapor, wherein the acetonitrile is recovered from the distillation column bottoms.

Preferably, the acetonitrile/water azeotrope leaving the column after the distillation of step A″ as a side draw, is in the form of a liquid.

Those impurities having a boiling point between the acetonitrile/water azeotrope boiling point and the acetonitrile boiling point, will be withdrawn in step A″ partly as vapor together with the low boiling impurities and partly as a liquid side draw together with the acetonitrile/water azeotrope. In step C″, depending on the distillation temperature selected and the exact boiling temperature of these impurities, they will mainly be withdrawn as vapor at the top of the column.

The distillations of steps A″ and C″ may be executed one after the other on the same column. However, it is preferable to perform them in separate columns. Hence, it is preferable to perform the distillation of step A″ in a first distillation column and the distillation of step C″ in a second distillation column.

According to the second embodiment of the invention, the reduction in water content of the stream fed to the column in which the distillation of step C″ is performed, is achieved by the use of pressure-swing distillation wherein the distillation of step A″ is performed at below atmospheric pressure. The percentage of water in the acetonitrile/water azeotrope is decreased at low pressure and increased at high pressure. Hence, under below atmospheric pressure conditions during the distillation of step A″, the water content in the acetonitrile/water azeotrope is decreased. Preferably, the pressure during the distillation of step A″ is between 150 and 400 mbar, and more preferably between 200 and 220 mbar and the azeotrope leaving the column after the distillation of step A″ as a side draw has a water content between 7.0% by weight and 13% by weight, and more preferably between 8.5 and 9.5% by weight.

Thereby it is ensured that the stream reaching the column, in which the distillation of step C″ is performed, has a reduced water content and is as rich as possible in acetonitrile. During the distillation of step C″, performed at atmospheric pressure, the water content of the azeotrope increases and reaches approximately 16% by weight. Hence, the remaining water can be distilled away in the form of a acetonitrile/water azeotrope. This azeotrope is preferably recycled to the feedstock for the distillation of step A″ or more preferably to the column slightly higher than the feed point of the raw material.

Low boiling impurities are defined as those impurities that have a lower boiling temperature than the acetonitrile/water azeotrope (76° C. at atmospheric pressure), or form an azeotrope with water that has a lower boiling temperature than the acetonitrile/water azeotrope. Examples are dichloromethane (boiling point: 40° C. at atmospheric pressure), acetone (boiling point: 56.2° C. at atmospheric pressure), methanol (boiling point: 64.5° C. at atmospheric pressure), diisopropylether (boiling point: 68° C. at atmospheric pressure), ethylacetate-water azeotrope (boiling point: 70.38° C. at atmospheric pressure) without being restricted thereto.

High boiling impurities are defined as those impurities that have a higher boiling temperature than acetonitrile (81.6° C. at atmospheric pressure). Examples are, isopropanol (boiling point 82.4° C. at atmospheric pressure), acetic acid (boiling point: 118.1° C. at atmospheric pressure), N,N-dimethylformamide (boiling point: 153° C. at atmospheric pressure), phenols, cresols without being restricted thereto.

The acetonitrile feedstock to be purified by the processes of the invention is preferably the waste from a HPLC purification process of peptides. The average composition of low grade acetonitrile stream resulting from an HPLC purification process of peptides is presented in the following Table:

TABLE 1

| Description | Composition | |
|---|---|---|
| Acetonitrile | 10-50% | by volume |
| Methanol | <1% | by volume |
| TFA | <0.1% | by weight |
| Acetone | <0.5% | by volume |
| Isopropanol | <0.5% | by volume |
| Acetic acid | <1.5% | by weight |
| NH₄OAc | <0.15% | by weight |
| Triethylamine Phosphate/ | <1% | by weight |
| H₃PO₄ (pH = 2) | | |
| Peptide residues | <0.1% | by weight |
| Ethylacetate, diisoproylether, | <0.1% | by weight (sum of all) |
| N,N-dimethylformamide, thiols, | | |
| phenols, cresols, | | |
| organohalogenated compounds, | | |
| aromatic compounds, silicated | | |
| compounds, other salts | | |

| | |
|---|---|
| Water | Balance |

Preferably, the water content in the feedstock is at least 50% by weight.

Depending on the properties of the acetonitrile feedstock, its pH is adapted with an acid or a base.

As explained above, according to both embodiments the high boiling impurities are produced as the column bottoms after the distillation of step A, A′ or A″. This bottom contains primarily water, traces of acetonitrile, salts and other impurities boiling at a temperature higher than 81.6° C. at atmospheric pressure; it may be considered as waste that can be treated in a biological wastewater plant.

According to both process embodiments outlined above, the feedstock fraction leaving the distillation column after the distillation of step A, A′ or A″ is small compared to the amount of the initial feedstock. If for example 20% by weight of the initial feedstock is acetonitrile, the acetonitrile/water azeotrope fraction leaving the column after the distillation in step A, A′ or A″ will only represent approximately 20×1.19 or 23.8% of the initial feedstock in the first embodiment using pervaporation and 20×1.11 or 22.2% of the initial feedstock in the second embodiment using pressure-swing distillation. This enables to keep the column sizes small and in particular to keep the pervaporation unit as small as possible.

According to the first embodiment of the invention, using pervaporation, it is possible to perform the step wherein the acetonitrile/water azeotrope and the low boiling impurities leaving the column after the distillation of step A or A′ are fed to a pervaporation unit, by first condensing the acetonitrile/water azeotrope and the low boiling impurities and then sending the condensate to the pervaporation unit.

Alternatively, it is also possible to perform the step wherein the acetonitrile/water azeotrope and the low boiling impurities leaving the column after the distillation of step A or A′ are fed to a pervaporation unit, by sending the acetonitrile/water azeotrope and the low boiling impurities as vapours under pressure over the pervaporation unit.

The permeate of the pervaporation unit, containing primarily water but also traces of acetonitrile and methanol, is preferably recycled to the acetonitrile feedstock and introduced into the column in which the distillation of step A or A′ takes place. The retentate of the pervaporation unit that is fed to the column, in which the distillation of step C or C′ takes place, contains primarily acetonitrile but contains also water (0.1 to 5%), traces of methanol, isopropanol, acetone and other low boiling impurities.

According to the first and second embodiment of the invention, the acetonitrile/water azeotrope is drawn as a vapor from the column in which the distillation of step C, C′ or C″ took place, and the acetonitrile is produced as the bottoms of this column. In some cases, this still contains some salts and high boiling hydrophobic impurities, such as toluene or xylenes. This happens, because such high boiling hydrophobic impurities can form hydrophobic-hydrophobic interactions with acetonitrile and thereby are dragged over the column top during distillation and also stay in the pervaporation retentate.

In order to purify the acetonitrile from these high boiling hydrophobic impurities, it is preferable to feed the column bottoms after the distillation of step C, C′ or C″ to a distillation column where they are again distilled. The pure acetonitrile is then drawn as vapor from the top of the column, in which the additional distillation was performed, and the evaporation residue is produced as column bottoms after this additional distillation.

The additional distillation may be performed in the same column where already the distillations of steps A, A′, A″ and /or C, C′, C″ were performed. However, it is preferable to use a separate column for this additional distillation.

The acetonitrile drawn from said column after the additional distillation is more than 99.8% pure.

However, in order to be used in HPLC, light transmittance of more than 90% at 220 nm and more than 98% at 240 nm must be achieved. Otherwise, the acetonitrile itself absorbs more light than the products present in the acetonitrile and the products would not be detectable by a UV detector.

Therefore, in a preferred aspect of both embodiments of the invention, the acetonitrile being produced as the column bottoms after the distillation of step C, C′ or C″ or, if applicable, the acetonitrile being drawn from the distillation column and being condensed after the additional distillation, is sent over a bed of activated carbon to render it into a HPLC grade acetonitrile. It is preferable to perform the additional distillation before the activated carbon bed is used since thereby rapid saturation of the activated carbon bed by salts and hydrophobic high boiling impurities is avoided.

The following examples are provided for illustrative purposes.

### EXAMPLE 1

This example is in accordance with the first embodiment of this invention and makes use of pervaporation.

The table below shows the results of 3 pilot trials performed on 3 different low-grade acetonitrile HPLC process wastes. The first HPLC process waste resulted from one single HPLC column, on which the purification was performed with a 0.05M Triethylamine phosphate/Acetonitrile buffer. The 2^{nd} one was a mixture of waste streams coming from several columns all working in different conditions. The 3^{rd} waste stream resulted also from one single column, but the purification was performed with 1% Acetic acid/Acetonitrile. The composition of the three waste streams is presented in the same table. Prior to the trials, all waste streams were neutralized to pH=6 with Na₂CO₃.

The different steps as presented in the process flow diagram of FIG. 1A, were conducted batch-wise one after the other. No side streams were recycled during these trials. All the distillations were performed in the same equipment: a 500 L reactor equipped with a distillation column with 7 theoretical plates. The 1^{st} azeotropic distillation was performed under atmospheric pressure with a reflux R of 0.5. This 1^{st} azeotropic distillation had to be performed twice, due to the volume of the reactor and due to the minimum amount of product needed in the pervaporation unit. In the pervaporation unit, a Sulzer PERVAP® 2201 plate membrane was used with a surface of 4 m². A vacuum of 30-40 mbar was applied on the permeate side, and the permeate was cooled to −10° C. to avoid re-evaporation. The incoming feed of the pervaporation unit was pressurised to 2-3 bar and heated to 90-95° C. In these conditions the flow was approximately 30 L/h. The retentate was then transferred to the distillation reactor again. First the remaining water was removed by distilling (with a reflux R of 0.83) a small head fraction over top (which was discarded), leaving an almost pure acetonitrile in the reactor bottoms. This acetonitrile was then redistilled completely. The results of step 3 in the table correspond to the results obtained after this final acetonitrile distillation. The redistilled acetonitrile was then finally passed over an activated carbon unit.

Typical specifications for HPLC-grade acetonitrile are:
Purity: not lower than 99.8%
Residue on evaporation: not more than 1.0 mg/L
Transmittance at 220 nm: >90%
Transmittance at 240 nm: >98%

TABLE 2

| Step | | Trial 1 | Trial 2 | Trial 3 |
|---|---|---|---|---|
| Incoming feedstock | | | | |
| Date | | 03-Oct-02 | 03-Oct-02 | 03-Oct-02 |
| Sample-ID | | ACN-2002-10-1 | ACN-2002-10-2 | ACN-2002-10-3 |
| Total weight, kg | | 941 | 914 | 974 |
| Analysis | | | | |
| H20, wt % (KF-method) | | 79.51 | 77.25 | 80.8 |
| AcCN, wt % (GC) | | 20.5 | 22.75 | 19.2 |
| Methanol, wt % (GC) | | — | — | — |
| Acetone, wt % (GC) | | — | — | — |
| Isopropanol, wt % (GC) | | — | — | — |
| pH | | 2.96 | 3.36 | 2.95 |
| pH after neutralization | | 6 | 6 | 6 |
| Transmittance (188-300 nm) | | Not OK | Not OK | Not OK |
| Step 1: azeotropic distillation | | | | |
| Date | | 07-Nov-02 | 22-Oct-02 | 25-Oct-02 |
| Start weight, kg | | 760 | 710.5 | 837 |
| Weight distillate, kg | | 156 | 202 | 160 |
| Sample-ID | | ACN-2002-10- | ACN-2002-10- | ACN-2002-10- |
| | | 1D1 | 2D1 | 3D1 |
| Analysis distillate: | | | | |
| H20, wt % (KF-method) | | 16.3 | 17.0 | 18.85 |
| pH | | 10 | 8.9 | 7.6 |
| Transmittance (188-300 nm) | | Not OK | Not OK | Not OK |
| Residue, mg/L | | 2 | 4 | 5 |
| Step 2: pervaporation | | | | |
| Date | | 12-Nov-02 | 24-Oct-02 | 30-Oct-02 |
| Start weight, kg | | 156 | 202 | 160 |
| Weight retentate, kg | | 83 | 153.5 | 101 |
| Sample-ID | | ACN-2002-10- | ACN-2002-10-2R | ACN-2002-10- |
| | | 1R | | 3R |
| Analysis distillate: | | | | |
| H20, wt % (KF-method) | | 2.2 | 2.38 | 2.3 |
| pH | | 10.8 | 9.8 | 9.8 |
| Transmittance (188-300 nm) | | Not OK | Not OK | Not OK |
| Step 3: 2nd and 3rd distillation | | | | |
| Date | | 14-Nov-02 | 06-Nov-02 | 05-Nov-02 |
| Start weight, kg | | 83 | 153.5 | 101 |
| Weight distillate, kg | | 51 | 52 | 53.5 |
| Sample-ID | | ACN-2002-10- | ACN-2002-10- | ACN-2002-10- |
| | | 1D2 | 2D2 | 3D2 |
| Analysis distillate: | | | | |
| H20, wt % (KF-method) | | 0.4 | 0.17 | 0.2 |
| pH | | 9.6 | 8.8 | 7.8 |
| Transmittance (188-300 nm) | | Not OK | Not OK | Not OK |
| Residue, mg/L | | <1 | <1 | <1 |
| Step 4: activated carbon | | | | |
| treatment | | | | |
| Date | | 19-Nov-02 | 08-Nov-02 | 14-Nov-02 |
| Start weight, kg | | 51 | 52 | 53.5 |
| Weight treated distillate, kg | | 42 | 31 | 35 |
| Sample-ID | | ACN-2002-10- | ACN-2002-10- | ACN-2002-10- |
| | | 1A2 | 2A2 | 3A2 |
| Analysis treated distillate: | Specs | | | |
| AcCN, wt % (GC) | >99.8% | 100 | 100 | 99.93 |
| Residue, mg/L | <1.0 mg/L | <1 | <1 | <1 |
| Transmittance (at 220 nm), in % | >90% | 95.3 | 96.6 | 95.1 |
| Transmittance (at 240 nm), in % | >98% | 99.8 | 100 | 98.7 |
| pH | | 11.5 | 8.2 | 7.3 |
| High boilers content, mg/L* | | not detected | not detected | not detected |

*HPLC detection limit: 100 mg/L

Sample-ID in the table above means Sample Identification number, wt % is an abbreviation for percent by weight and GC means gas chromatography. Specs means the typical specifications for HPLC-grade acetonitrile as defined above.

KF-method is an abbreviation for Karl Fischer method, a standardised titration method for water-content determination, see N. D. Cheronis and T. S. Ma, Organic Functional Group Analysis (Wiley-Interscience, New York, 1964), pp. 472-475. An extensive study of the method can be found in J. Mitchell and D. Smith, Aquametry (Wiley-Interscience, New York, 1948).

### RESULTS

These results clearly show that the process according to the first embodiment of the invention allows recovery of HPLC grade acetonitrile from low grade acetonitrile feedstocks comprising water content of 77% and more.

### EXAMPLE 2

This example is in accordance with the second embodiment of this invention and makes use of pressure-swing distillation.

In this example, the waste stream was neutralized to pH=6.4 with Na₂CO₃. Tables 3, 4 and 5 below set forth the conditions of a lab trial of the pressure-swing process. Since only one distillation column was available, the first two distillations of the process represented in FIG. 2, were executed one after the other on the same column. A glass bubble cap tray column, purchased from Normag Germany, was used with a diameter of 50 mm and with 30 bubble cap trays. A bubble cap column is a column with trays that possesses bubble caps through which the vapours pass into the liquid on the tray. The gas flows up through a center riser and cap, and finally passes into the liquid through a series of openings or slots in the lower side of the cap. The device has a built-in seal that prevents liquid drainage at low gas-flow rates.

To simulate the recycle stream coming from the top of the the 2^{nd} column into the feedstock of the first low pressure distillation, a mixture was made of an industrial low grade acetonitrile waste resulting from an HPLC purification process of peptides and containing 0.029% by weight methanol, 0.009% by weight acetone, 0.017% by weight isopropanol, 16,873% by weight acetonitrile, 0.270% by weight benzoylchloride and 82.8% by weight water, with a 2^{nd} stream that was obtained by an azeotropic distillation at atmospheric pressure of the low grade acetonitrile. This 2^{nd} stream was obtained in a separate distillation prior to the preparation of mixture and contained 0.059% by weight methanol, 0.034% by weight ethanol, 0.042% by weight acetone, 0.152% by weight isopropanol, 84.12% by weight acetonitrile and 15.6% by weight water. The recycle stream mass fraction in the mixture was approximately 46%, resulting in a feedstock for the 1^{st} column containing 51.5% H₂O.

The above prepared mixture was fed into the 1^{st} distillation column on tray 21, the lowest tray being tray 30 and the highest tray being tray 1. The feed temperature was 38.1° C. The distillation was operated at a pressure of 300 mbar, with a reflux ratio R/D (Reflux/Distillate) of 40/1. This means that for 41 parts undergoing distillation and condensation, 40 parts are sent back into the column and 1 part is withdrawn as distillate. The low boiling impurities, leaving the column via the top, were first condensed at 10° C. This first condensate is indicated as “distillate” in table 2. The vapours that were not yet condensed in this first condenser were passed over a 2^{nd} condenser at 2° C. This 2^{nd} condensate is indicated as “distillate 2” in table 1. The AcCN—H₂O azeotrope (approximately 88 percent weight of AcCN/12% percent weight of H₂O) was withdrawn as a side draw on tray 11, and indicated as “side product” in table 2.

This side draw was collected and then later on fed to the same column as the one used in the first distillation. The feed was entered into the column on tray 11. The feed temperature was 43° C. The distillation was operated at atmospheric pressure, with a reflux ratio R/D of 1/1. The water was withdrawn as AcCN—H₂O azeotrope via the top of the column, leaving an almost completely dewatered AcCN in the bottom, containing 0.02 percent by weight of H₂O.

This bottom fraction was then distilled in another distillation column to get rid of the high boiling impurities. For this a glass 15 mm packed column was used with 40 theoretical plates. The feed position was the bottom in this case, since this lab column did not have the possibility to enter the feed at the side. The distillation was performed at atmospheric pressure, with a reflux ratio R/D of 3/1, giving a pure acetonitrile at the top and leaving the high boiling components in the bottom. The column bottoms contained 99.16% by weight acetonitrile, 0.67% by weight propionitrile and 0.160% n-butylacetate.

The pure acetonitrile distillate was then passed over an active carbon bed with a diameter of 30 mm and a length of 1 m, to obtain an HPLC grade acetonitrile. The final product had a GC (gas chromatography) purity of 100 percent weight. The residue on evaporation was 0 mg/l. UV transmittance at 220 nm was 91.7%; UV transmittance at 240 nm was 99.5%.

TABLE 3
Experimental steady-state conditions of the first column (low pressure)

| | | | | | Temperature (T = Tray from the top) | | | | | | | | | | | | |
|---|---|---|---|---|---|---|---|---|---|---|---|---|---|---|---|---|---|
| Mass flow | | | | | | | | | | | | T 11 | | | | | |
| | Side- | Dis- | Distillate | | | Bot- | | | Feed- | | | Side- | | | | Reflux | Pressure |
| Feed | product | tillate | 2¹⁾ | Bottom²⁾ | Feed | tom | unter | T 24 | point | T 18 | T 14 | prod | T 8 | T 4 | Top | ratio | at top |
| [kg/h] | [kg/h] | [kg/h] | [kg/h] | [kg/h] | [° C.] | [° C.] | 1 | [° C.] | T 21 | [° C.] | [° C.] | [° C.] | [° C.] | [° C.] | [° C.] | [R/D] | [mbar] |
| 0.860 | 0.420 | 0.027 | 0.002 | 0.411 | 38.1 | 71.7 | 71.7 | 70.4 | | 46.1 | 45.3 | 44.8 | 44.9 | 44.3 | 44.4 | 40/1 | 300 |

Distillate 2 = Distillate after the condenser (aftercooler 2° C.)Bottom product = Feed − Distillate − Distillate 2
Column: “Normag” - bubble-cap tray column (50 mm) in glass with 30 trays (tray efficient 70-80%)
Feed position: Tray 21 from the top

TABLE 4
Experimental steady-state conditions of the second column (high pressure)

| | | | | Temperature (T = Tray from the top) | | | | | | | | | | | | |
|---|---|---|---|---|---|---|---|---|---|---|---|---|---|---|---|---|
| | | | | | | | | | | | T 11 | | | | Reflux | |
| Feed | Distillate | Distillate 2¹⁾ | Bottom²⁾ | Feed | Bottom | | T 24 | T 21 | T 18 | T 14 | Feed- | T 8 | T 4 | Top | ratio | Pressure |
| [kg/h] | [kg/h] | [kg/h] | [kg/h] | [° C.] | [° C.] | unter 1 | [° C.] | [° C.] | [° C.] | [° C.] | point | [° C.] | [° C.] | [° C.] | [R/D] | [mbar] |
| 0.840 | 0.580 | 0.002 | 0.258 | 42.8 | 82.0 | | 80.4 | 78.3 | 76.8 | 76.2 | | 76.1 | 75.8 | 77.4 | 1:1 | 1013 |

¹⁾Distillate 2 = Distillate after the condenser (aftercooler 2° C.)
²⁾Bottom product = Feed − Distillate − Distillate 2

TABLE 5
Experimental steady-state conditions of the third column

| | | | | | Reflux | |
|---|---|---|---|---|---|---|
| Feed | Distillate | Bottom²⁾ | Bottom | Top | ratio | Pressure |
| [kg/h] | [kg/h] | [kg/h] | [° C.] | [° C.] | [R/D] | [mbar] |
| 0.026 | 0.024 | 0.002 | 82.0 | 82.0 | 3:1 | 1013 |

### RESULTS

These results clearly show that the process according to the second embodiment of the invention allows recovery of HPLC grade acetonitrile from low grade acetonitrile feedstocks comprising a water content of 51.5% and more.

1. A process for purifying an acetonitrile feedstock comprising acetonitrile, 16% up to 90% by weight of water, low boiling impurities having a boiling temperature lower than the acetonitrile/water azeotrope boiling point, and high boiling impurities having a boiling temperature higher than the acetonitrile boiling point, the process comprising in sequence the steps of:
A) introducing the feedstock into a distillation column and, by performing a distillation, separating the acetonitrile/water azeotrope and the low boiling impurities from the high boiling impurities, the acetonitrile/water azeotrope and the low boiling impurities being drawn as a vapor from the top of said distillation column, the high boiling impurities being produced as the distillation column bottoms;
B) feeding the acetonitrile/water azeotrope and the low boiling impurities to a pervaporation unit capable of separating the water from the acetonitrile, the majority of water being collected as permeate from the pervaporation unit, and the acetonitrile, low boiling impurities and remaining water being collected as retentate;
C) introducing the retentate of the pervaporation unit into a distillation column and, by performing another distillation, withdrawing the remaining acetonitrile/water azeotrope and the low boiling impurities as a vapor, wherein the acetonitrile is recovered from the distillation column bottoms.

2. A process for purifying an acetonitrile feedstock comprising acetonitrile, 16% up to 90% by weight of water, low boiling impurities having a boiling temperature lower than the acetonitrile/water azeotrope boiling point, and high boiling impurities having a boiling temperature higher than the acetonitrile boiling point, the process comprising in sequence the steps of:
A′) introducing the feedstock in to a distillation column and, by performing a distillation, separating the acetonitrile/water azeotrope and the low boiling impurities from the high boiling impurities, the high boiling impurities being produced as the distillation column bottoms, a majority of the low boiling impurities being drawn as a vapor from the top of said distillation column, and the acetonitrile/water azeotrope and a remaining part of the low boiling impurities being drawn as a side draw of that distillation column;
B′) feeding the acetonitrile/water azeotrope and the low boiling impurities to a pervaporation unit capable of separating the water from the acetonitrile, the majority of water being collected as permeate from the pervaporation unit, and the acetonitrile, low boiling impurities and remaining water being collected as retentate;
C′) introducing the retentate of the pervaporation unit into a distillation column and, by performing another distillation, withdrawing the acetonitrile/water azeotrope and the low boiling impurities as a vapor, wherein the acetonitrile is recovered from the distillation column bottoms.

3. A process according to claim 2 wherein the acetonitrile/water azeotrope leaving the column, in which the distillation of step C′ is performed, is recycled to the acetonitrile feedstock and introduced into the distillation column, in which the distillation of step A′ is performed.

4. A process according to claim 1, wherein the distillation of step A or A′ is performed in a first distillation column and the distillation of step C or C′ is performed in a separate second distillation column.

5. A process according to claim 1 wherein the step, in which the acetonitrile/water azeotrope and the low boiling impurities leaving the column after the distillation of step A or A′ are fed to a pervaporation unit, is performed by first condensing the acetonitrile/water azeotrope and the low boiling impurities and then sending the condensate to the pervaporation unit.

6. A process according to claim 1 wherein the step, in which the acetonitrile/water azeotrope and the low boiling impurities leaving the column after the distillation of step A or A′ are fed to a pervaporation unit, is performed by sending the acetonitrile/water azeotrope and the low boiling impurities as vapours under pressure over the pervaporation unit.

7. A process according to claim 1 wherein the permeate of the pervaporation is recycled to the acetonitrile feedstock and introduced into the distillation column in which the distillation of step A or A′ takes place.

8. A process for purifying an acetonitrile feedstock comprising acetonitrile, 16% up to 90% by weight of water, low boiling impurities having a boiling temperature lower than the acetonitrile/water azeotrope boiling point, and high boiling impurities having a boiling temperature higher than the acetonitrile boiling point, the process comprising in sequence the steps of:
A″) introducing the feedstock into a distillation column and, by performing a distillation at below atmospheric pressure, separating the azeotrope and the low boiling impurities from the high boiling impurities, the high boiling impurities being produced as distillation column bottoms, the total or majority of low boiling impurities being drawn as vapor via the top of the distillation column and the acetonitrile/water azeotrope and potentially remaining low boiling impurities being drawn as a side draw of that distillation column;
C″) introducing the acetonitrile/water azeotrope side draw into a distillation column and, by performing another distillation, at atmospheric pressure, enriching the acetonitrile/water azeotrope with water and withdrawing it as a vapor, wherein the acetonitrile is recovered from the distillation column bottoms.

9. A process according to claim 8, wherein the distillation of step A″ is performed in a first distillation column and the distillation of step C″ is performed in a separate second distillation column.

10. A process according to claim wherein the pressure during the distillation of step A″ is between 150 and 400 mbar, and more preferably between 200 and 220 mbar and the azeotrope leaving the column after the distillation of step A″ as a side draw has a water content between 7.0% by weight and 13% by weight, and more preferably between 8.5 and 9.5% by weight.

11. A process according to claim 8 wherein the acetonitrile/water azeotrope leaving the column, in which the distillation of step C″ is performed, is recycled to the acetonitrile feedstock and introduced into the distillation column, in which the distillation of step A″ is performed.

12. A process according to claim 1, wherein the water content in the feedstock is at least 50% by weight.

13. A process according to claim 1, wherein the pH of the feedstock, before being introduced into the distillation column to perform the distillation of step A, A′ or A″ is adapted with an acid or base.

14. A process according to claim 1, wherein the distillation column bottoms produced after the distillation of step C, C′ or C″, containing the acetonitrile, are distilled once more in a distillation column, the pure acetonitrile being drawn as vapor from the distillation column and the evaporation residue being produced as column bottoms after this additional distillation.

15. A process according to claim 1, wherein the acetonitrile being produced as the column bottoms after the distillation of step C, C′ or C″ or the pure acetonitrile being drawn as vapor and condensed after the additional distillation, are sent over a bed of activated carbon.
